(19) United States
(12) Patent Application Publication
(10) Pub. No.: US 2008/0159987 A1
(43) Pub. Date: Jul. 3, 2008

(54) Use of Rifaximin for the Treatment of Restless Legs Syndrome

(76) Inventor: Leonard Weinstock, St. Louis, MO (US)

Correspondence Address: HUSCH BLACKWELL SANDERS LLP, 190 CARONDELET PLAZA, SUITE 600, ST. LOUIS, MO, 63105-3441, US

(21) Appl. No.: 11/619,521

(22) Filed: Jan. 3, 2007

(51) Int. Cl. A61K 31/437; A61K 9/36; A61K 33/30; A61K 35/74; A61P 25/00

(52) U.S. Cl. 424/93.4; 514/294; 424/479; 424/641

(57) ABSTRACT

Restless legs syndrome (RLS) is characterized by an urge for leg movement, often with abnormal leg sensations. Symptoms are triggered by rest, often at night, and improve temporarily with movement, especially walking. This syndrome has a prevalence ranging from 7% to 15% and significantly contributes to sleep disorders. A method of prevention of restless legs syndrome is disclosed that comprises orally administering a composition containing a therapeutically effective amount of rifaximin to a patient exhibiting symptoms of restless legs syndrome.

### CROSS REFERENCES

None.

### TECHNICAL FIELD OF THE INVENTION

The present invention relates generally to the medical field and, more particularly, to the use of rifaximin in the treatment of restless legs syndrome.

### BACKGROUND OF THE INVENTION

In general, rifaximin is well known as a non-systemic antibiotic (<0.4%) characterized by activity against a broad spectrum of enteric bacterial pathogens and the delivery of high concentrations of antibiotic to the gastrointestinal tract.

The antibiotic rifaximin was discovered in 1980 and originally patented in Italy as IT Patent 1154655 granted on Jan. 21, 1987. The related U.S. Pat. No. 4,341,785 to Marchi et al. discloses imidazo-rifamicyn derivatives having antibacterial utility, and the related process for preparing it. The '785 patent also discloses a pharmaceutical antibacterial composition and a method of using it to treat antibacterial diseases of the gastrointestinal tract. A further patent, U.S. Pat. No. 4,557,866 to Cannata et al. discloses a process for the synthesis of pyrido-imidazo rifamycins. The process is described as an improvement over the '785 patent to Marchi in that the later process provides unsatisfactory yields from an industrial point of view. The entire disclosures of the '785 and '866 patents are incorporated by reference herein.

Rifaximin is essentially a non-absorbable semi-synthetic antibiotic, related to rifamycin. The antimicrobial spectrum (in vitro) includes most gram-positive and gram-negative bacteria; and both aerobes and anaerobes.

It presents low risk for drug interactions (no effect on drugs metabolized by cytochrome p450 enzyme system) and about the same adverse properties as compared to a placebo. When ingested in tablet or pill form rifaximin is concentrated in the gastrointestinal tract and primarily excreted unchanged in the feces. It binds to the beta subunit of bacterial DNA-dependent RNA polymerase, which inhibits bacterial RNA synthesis. In contrast with other antibiotics, resistance to rifaximin is not plasmid-mediated but utilizes a chromosomal one-step alteration in the DNA-dependent RNA polymerase. In subjects using rifaximin no relevant resistance has been observed. Further, mutant resistant bacteria showed reduced viability and there is no systemic cross resistance for rifampin.

Since rifaximin is practically insoluble in water and is non absorbed (<0.4%) after oral administration, it can be used to treat localized diseases of the gastrointestinal tract. Rifaximin products specific for enteric pathogens of the gastro-intestinal tract are presently commercially marketed under various trade names—NORMIX® available from Alfa Wassermann S.p.A., Bologna, Italy; XIFAXAN® available from Salix Pharmaceutical, Raleigh, N.C.; REDACTIV® available from GlaxoSmithKline and FLONORM® from Schering-Plough. Since the solubility of rifaximin in water is approximately 1 .mu.gmL.sup.3 the drug is virtually undissolved when traveling through the GI tract. The relative insolubility of rifaximin is thought to influence bacterial susceptibility and subsequent eradication due to the invasive nature of some enteric pathogens (e.g. Salmonella and Campylobacter). The relative insolubility of rifaximin also leads to its negligible systemic absorption. Rifaximin has been known to be effective for treating infections that are localized to the gut and is not known to be suitable for treating systemic infections caused by invasive organisms.

Rifaximin has been marketed in Italy since 1985 under the trademark NORMIX® for treating acute and chronic intestinal infections from gram-positive and gram-negative bacteria and as adjuvant in the therapy of the hyperammonoaemia. At present NORMIX® is marketed in the shape of pharmaceutical compositions, orally administrable, made by tablets or by granulates containing suitable pharmaceutically acceptable excipients together with rifaximin, but also other pharmaceutical forms orally administrable like capsules, sugar coated tablets and syrups can be used. XIFAXAN® is marketed in the United States and Canada and includes rifaximin as the active ingredient. The formulation is used in the treatment of travelers' diarrhea caused by the noninvasive strains of Escherichia coli. XIFAXAN® is a non absorbable antibiotic for gastrointestinal infections. Products similar to NORMIX® and XIFAXAN® are marketed in Mexico under the tradenames REDACTIV® and FLONORM®.

Other uses of rifaximin are disclosed in the following patents:

U.S. Pat. No. 5,886,002 to Ferrieri et al. describes use of rifaximin compositions in the treatment of diarrhea from cryptosporidiosis.

U.S. Pat. No. 5,352,679; to Ferrieri et al. describes use of rifaximin (INN) in formulations for treatment of gastric dyspepsia caused by Helicobacter pylori bacteria.

U.S. Pat. Nos. 5,314,904 and 6,140,355 both to Egidio et al. disclose compositions containing rifaximin for treatment of vaginal infections.

Known therapeutic uses of rifaximin, administered in a tablet form, include Clostridum difficile-associated diarrhea, Crohn's disease, Diverticular disease, Hepatic encephalopathy, Helicobacter pylori eradication, infectious diarrhea, irritable bowel syndrome, pouchitis, prophylaxis for GI surgery, small bowel overgrowth, traveler's diarrhea and ulcerative colitis. These therapies are directed to pediatric, adult and elderly subjects.

The use of rifaximin in the treatment of restless legs syndrome has never been investigated.

Restless legs syndrome (RLS) is characterized by an urge for leg movement, often with abnormal leg sensations. Symptoms are triggered by rest, often at night, and improve temporarily with movement, especially walking. This syndrome has a prevalence ranging from 7% to 15% and significantly contributes to sleep disorders. Most RLS cases are idiopathic, but contributing factors include iron deficiency and renal failure. Restless legs syndrome occurs frequently in patients with peripheral neuropathy, particularly in individuals with Charcot-Marie-Tooth disease. The prevalence of RLS has also been reported in a single case series of fibromyalgia (31% of 135 patients) and scleroderma (22% of 27 patients).

It would be desirable to provide an effective treatment for restless legs syndrome.

The present invention is directed to meeting one or more of the above-stated desirable objectives.

### SUMMARY OF THE INVENTION

The present invention provides a method of treating restless legs syndrome in a human comprising administration of rifaximin.

In general, in the method of the invention, the therapeutically effective amount of rifaximin delivers a dosage to achieve a concentration of approximately 400 mg per application. It is believed that dosages in the concentration range between about 400 mg and about 800 mg per application three times a day would also be effective. The duration of treatments with the invention formulations can be at daily, weekly or monthly intervals depending on the individual and the desired outcome. However, it is preferred that applications be administered three times daily for at least ten days.

Although this disclosure is directed to the preferred use of rifaximin, it is also within the scope of the invention that any broad spectrum antibiotic can be included in the compositions and are included herein.

These and other objects, aspects, features and advantages of the present invention will become apparent from the following detailed description when taken in conjunction with the referenced drawings.

### BRIEF DESCRIPTION OF THE DRAWINGS

Not Applicable.

### DETAILED DESCRIPTION

Substantial overlap exists between irritable bowel syndrome (IBS) and other hypersensitivity disorders. Small intestinal bacterial overgrowth (SIBO) may be a common denominator for these disorders, which are characterized by visceral hypersensitivity, chronic inflammation, and neurochemical changes. Altered genetic control of immune activation, production of proinflammatory cytokines, increased intestinal permeability, and immune responses triggered by bacterial endotoxins may lead to the development of intestinal and extraintestinal manifestations of IBS. Persistence of an underlying motility disorder of the migrating motor complex is thought to be responsible for SIBO and GI symptom relapse after antibiotic therapy.

It is hypothesized that a relationship might exist between restless legs syndrome (RLS), IBS, and SIBO. Post-infectious IBS is related to SIBO. Evidence that SIBO plays a role in IBS continues to accumulate despite continued debate and controversy. In a single case series, patients with scleroderma had an increased incidence of RLS, although they did not have end-stage small intestinal disease with the commonly accepted presentation of SIBO, such as malabsorption and steatorrhea. Another possible mechanism to explain a role for SIBO in RLS is that a chronic inflammatory state directly and indirectly (via increasing hepcidin levels) plays a role in iron deficiency, which is sometimes associated with RLS. Iron deficiency can affect cell oxygenation by direct (e.g., reduced hemoglobin levels) and indirect mechanisms (e.g., functioning of mitochondrial iron-containing proteins required to process oxygen in cells). It is theorized that the latter may be one mechanism to explain RLS muscle discomfort that is relieved by leg movement.

An embodiment of the present invention provides a treatment for RLS comprising oral administration of a therapeutically effective amount of rifaximin. A dosage of 400 mg per application three times daily is indicated as therapeutically effective. It is believed that dosages in the concentration range between 400 mg and 800 mg per application three times a day would also be effective. This course of treatment is advantageously continued for a period of at least ten days. Rifaximin may be administered in a number of forms, which are disclosed and described in U.S. Pat. Nos. 4,341,785; 5,886,002; 5,352,679; 5,314,904; 6,140,355; and 7,045,620 and U.S. Publication No. 2006/0210592, the entire disclosures of which are expressly incorporated by reference herein.

In an alternate embodiment, a course of treatment with rifaximin is followed by long-term administration of the prokinetic medicine tegaserod (for example, ZELNORM® produced by Novartis Pharmaceuticals Corporation, East Hanover, N.J.) 3 mg nightly.

In yet another embodiment, the course of treatment with rifaximin is followed by short-term treatment with zinc 220 mg/d for 1 month for increased small intestinal permeability and a bifidobacteria-based probiotic (for example, FLORA-Q® produced by Kenwood Therapeutics, Fairfield, N.J.) once daily for one month. This short term administration of zinc and a bifidobacteria-based probiotic may be combined with the previously described follow up administration of tegaserod.

The biological example provided herein provides support for the novel hypotheses that SIBO associated with IBS may be an important factor in some patients with RLS and that comprehensive SIBO therapy may provide long-term improvement in both RLS and GI symptoms.

### Biological Example

A patient had a 14-year history of chronic postprandial abdominal pain and diarrhea that had immediately followed an episode of travelers' diarrhea, with subsequent development of RLS two months later. It was suspected that the patient might have postinfectious IBS and SIBO. Empiric treatment with the minimally absorbed (<0.4%), broad-spectrum antibiotic rifaximin resulted in a dramatic, rapid, and prolonged response for both RLS and IBS symptoms. In an open-label, observational pilot trial, the efficacy of rifaximin, followed by treatment for possible motility and permeability disturbance, was evaluated in IBS patients with RLS who showed indirect evidence of SIBO.

Patients visiting a community-based adult gastroenterology clinic over a 4-month period were eligible for the study if they presented with functional bowel symptoms and had abnormal lactulose breath test (LBT) results. Patients were excluded if they had celiac disease, Crohn's disease, pancreatic insufficiency, ulcerative colitis, scleroderma, diabetes, chronic renal failure, or surgeries predisposing them to SIBO. A diagnosis of RLS was confirmed using the Johns Hopkins validated interview process.

Patients consumed a low carbohydrate dinner and fasted overnight. Breath samples were collected every 15 minutes for 180 minutes and were analyzed by gas chromatography (using, for example, a QUINTRON DP PLUS MICROLYZER™ manufactured by QuinTron Instrument Company, Milwaukee, Wis.) for levels of hydrogen and methane. After a baseline recording, patients consumed 10 g of lactulose powder (for example, KRISTALOSE® produced by Bertek Pharmaceuticals Inc, Sugar Land, Tex.) dissolved in 240 mL of water. An abnormal LBT result was defined as an increase of greater than 20 ppm above baseline levels for hydrogen and/or methane gas 180 minutes or less after ingesting lactulose. An abnormal flatline LBT result was defined as a lack of a greater than 5-ppm increase above baseline levels for either hydrogen or methane during the 180-minute test. The role of SIBO in IBS and diseases other than restless legs syndrome, as well as the efficacy of breath tests in the diagnosis of SIBO is discussed in U.S. Pat. Nos. 6,861,053; 6,805,852; 7,056,686; and 7,048,906 to Lin and Pimentel, the disclosures of each of which are expressly incorporated by reference herein.

Patients received rifaximin (XIFAXAN® produced by Salix Pharmaceuticals, Inc, Morrisville, N.C.) 400 mg three times daily for ten days. This was followed by long-term administration of the prokinetic medicine tegaserod (ZELNORM® produced by Novartis Pharmaceuticals Corporation, East Hanover, N.J.) 3 mg nightly plus short-term treatment with zinc 220 mg/d for one month for increased small intestinal permeability and a bifidobacteria-based probiotic (FLORA-Q® produced by Kenwood Therapeutics, Fairfield, N.J.) once daily for one month. Two patients were treated with rifaximin alone.

Each patient completed a questionnaire identifying the occurrence and rating the severity of six IBS symptoms experienced during the week before the LBT was conducted (baseline): abdominal pain, bloating, constipation, diarrhea, flatulence, and postprandial fullness. A history consistent with RLS and fibromyalgia was sought. Serum ferritin levels were measured at baseline.

Each patient received a follow-up questionnaire by mail and was asked to report on each of the gastrointestinal (GI) symptoms originally reported to be present at baseline as well as the percentage of improvement immediately after completing rifaximin treatment (day 11) and at the end of follow-up. Patients rated overall GI symptom improvement at the last follow-up visit as greatly improved, moderately improved, mildly improved, or no improvement. The percentage of RLS symptom improvement was assessed at the end of follow-up.

Thirteen IBS patients with confirmed RLS were included in the study. Eleven of these 13 patients were originally included in a SIBO study of IBS: 19 of 161 patients with an abnormal LBT result thought they had RLS. A diagnosis of RLS was confirmed in 11 of these 19 patients by the Johns Hopkins validated interview process. The remaining 2 of 13 patients with RLS (1 was the propositus and the other was referred from a neurology clinic) had been previously evaluated and treated for RLS by a neurologist.

All 12 patients who were tested for SIBO had an abnormal LBT result. The propositus was treated empirically. Most of these 12 patients (75%) were diagnosed with high-hydrogen levels. One of these patients had a flatline LBT result. This result suggested the possibility of overgrowth of hydrogen sulfide-producing bacteria in the small intestine, which is not detectable by current LBT technology. The most common GI symptoms at baseline included abdominal pain (n=13), bloating (n=13), diarrhea (n=11), and constipation (n=9).

Eleven patients were treated with rifaximin 1200 mg/d for ten days, followed by treatment with a prokinetic, zinc for intestinal permeability, and probiotic therapy. The remaining two patients (the neurology clinic patient and propositus) received a modified treatment regimen. The neurology patient was treated for ten days with rifaximin 400 mg three times daily for ten days. The propositus was treated empirically (prior to LBT availability at the clinic) and received rifaximin 400 mg three times daily for ten days and a second course two months post-treatment. Two months after completion of the second course, the patient was prescribed rifaximin 800 mg/d for twelve months.

Only 3 of 13 patients were diagnosed with possible iron deficiency at baseline. The neurology patient had borderline iron deficiency (ferritin level, 12 ng/dL; normal, <10 ng/dL) and received concomitant long-term iron supplementation (ferrous sulfate 325 mg 3 times daily). One additional patient with borderline normal levels of ferritin and one patient with low ferritin levels did not receive iron supplementation during SIBO therapy.

Percentages of RLS and IBS symptom improvement as well as global IBS symptom improvement were assessed at a mean of 107 days (range, 22-450 days). Ten of 13 patients with RLS exhibited at lest 80% improvement in RLS symptoms at the last follow-up visit. The ten patients who responded included the three patients who had borderline or low serum levels of iron at baseline. All ten patients indicated that the at least 80% improvement in RLS symptoms occurred during or at the end of rifaximin treatment. Eight of the ten patients with at least 80% improvement in RLS symptoms were followed long-term (mean, 139 days; range, 54-450 days), and 5 of the 8 patients reported complete (100%) resolution of RLS symptoms at the last follow-up visit.

After completion of ten days of rifaximin therapy (day 11), GI symptom improvement was reported as follows: abdominal pain (74%), diarrhea (73%), bloating (70%), postprandial fullness (65%), constipation (64%), and flatulence (47%). At the end of follow-up, symptom improvement was maintained: abdominal pain (81%), diarrhea (68%), bloating (76%), postprandial fullness (67%), constipation (59%), and flatulence (55%). Global symptom improvement was rated as greatly improved for 6 patients, moderately improved for five patients, and mildly improved for two patients. Follow-up LBT was not available for the 13 patients, with the exception of the neurology patient. This individual had a normal LBT result two months after completing rifaximin therapy.

Substantial overlap exists between IBS and other hypersensitivity disorders. Small intestinal bacterial overgrowth may be a common denominator for these disorders, which are characterized by visceral hypersensitivity, chronic inflammation, and neurochemical changes. Altered genetic control of immune activation, production of proinflammatory cytokines, increased intestinal permeability, and immune responses triggered by bacterial endotoxins may lead to the development of intestinal and extraintestinal manifestations of IBS. Persistence of an underlying motility disorder of the migrating motor complex is thought to be responsible for SIBO and GI symptom relapse after antibiotic therapy.

It is hypothesized that a relationship might exist between RLS, IBS, and SIBO. Evidence that SIBO plays a role in IBS continues to accumulate despite continued debate and controversy. In a single case series, patients with scleroderma had an increased incidence of RLS, although they did not have end-stage small intestinal disease with the commonly accepted presentation of SIBO, such as malabsorption and steatorrhea. Another possible mechanism to explain a role for SIBO in RLS is that a chronic inflammatory state directly and indirectly (via increasing hepcidin levels) plays a role in iron deficiency, which is sometimes associated with RLS. Iron deficiency can affect cell oxygenation by direct (e.g., reduced hemoglobin levels) and indirect mechanisms (e.g., functioning of mitochondrial iron-containing proteins required to process oxygen in cells). It is theorized that the latter may be one mechanism to explain RLS muscle discomfort that is relieved by leg movement.

The biological example provided herein provides support for the novel hypotheses that SIBO associated with IBS may be an important factor in some patients with RLS and that comprehensive SIBO therapy may provide long-term improvement in both RLS and GI symptoms.

Other objects, features and advantages of the present invention will be apparent to those skilled in the art. While preferred medications, uses and steps of the method have been illustrated and described, this had been by way of illustration and the invention should not be limited except as required by the scope of the appended claims.

1. A method of treating restless legs syndrome comprising orally administering a composition containing a therapeutically effective amount of rifaximin to a patient exhibiting symptoms of restless legs syndrome.

2. The method set forth in claim 1, wherein said composition is a pharmaceutical composition.

3. The method set forth in claim 1, wherein said therapeutically effective amount of rifaximin is about 400 mg per application.

4. The method set forth in claim 1, wherein said therapeutically effective amount of rifaximin is in the range of about 400 mg to about 800 mg per application.

5. The method set forth in claim 3, wherein said method further comprises three applications of rifaximin daily for a period of at least ten days.

6. The method set forth in claim 1, wherein said composition is selected from tablets, capsules, sugar coated tablets or syrups.

7. The method set forth in claim 1, wherein said composition is a liquid preparation.

8. The method set forth in claim 1, wherein said composition is a dispersable or disintegrating tablet.

9. The method set forth in claim 1, further comprising the administration of a prokinetic medicine to said patient.

10. The method set forth in claim 9, wherein said prokinetic medicine is tegaserod.

11. The method set forth in claim 1, further comprising the administration of zinc to said patient.

12. The method set forth in claim 10, wherein about 220 mg per day of zinc are administered to said patient for a period of at least one month.

13. The method set forth in claim 1, further comprising the administration of a bifidobacteria-based probiotic.

14. The method set forth in claim 12, wherein said bifidobacteria-based probiotic is administered to said patient once daily for at least one month.

15. The method set forth in claim 1, further comprising the step of conducting a lactulose breath test on said patient prior to administrating said therapeutically effective amount of rifaximin.

16. A method of treating restless legs syndrome, comprising:
orally administering a composition containing a therapeutically effective amount of rifaximin to a patient exhibiting symptoms of restless legs syndrome;
administering a prokinetic medicine to said patient; and
administering a bifidobacteria-based probiotic to said patient.

17. The method set forth in claim 16, wherein said prokinetic medicine and said bifidobacteria-based probiotic are administered to said patient following administration of rifaximin.

18. A method of treating restless legs syndrome, comprising:
conducting a lactulose breath test on a patient exhibiting symptoms of restless legs syndrome;
orally administering a composition containing a therapeutically effective amount of rifaximin to said patient;
administering a prokinetic medicine to said patient; and
administering a bifidobacteria-based probiotic to said patient.
